(19) United States
(12) Patent Application Publication
(10) Pub. No.: US 2019/0350456 A1
(43) Pub. Date: Nov. 21, 2019

(54) Portable Eye Pressure Sensor

(71) Applicant: Jeffrey E. Koziol, Arlington Heights, IL (US)

(72) Inventor: Jeffrey E. Koziol, Arlington Heights, IL (US)

(21) Appl. No.: 16/528,778

(22) Filed: Aug. 1, 2019

Related U.S. Application Data
(63) Continuation-in-part of application No. 15/986,953, filed on May 23, 2018.
(60) Provisional application No. 62/509,888, filed on May 23, 2017.

(51) Int. Cl. A61B 3/16

(52) U.S. Cl. CPC A61B 3/16

(57) ABSTRACT

Systems and methods for measuring IOP including a tonometer comprising sensor for detecting corneal contacted area and outputting an indication thereof to an observer to facilitate determination of when an endpoint is reached such that certain area of cornea has been flattened to obtain correct IOP reading. Camera and/or an image sensor can be provided for real-time video streaming or still frame photo imaging of retina as cornea flattens during measurement procedure. Independently of, or in conjunction with, sensing of corneal contact area, real-time or post processing of image data can be performed to determine when the endpoint is reached to obtain correct IOP reading.

This application is a continuation in part application of U.S. patent application Ser. No. 15/986,953, filed May 23, 2018, which claims priority to prior U.S. Provisional Patent Application No. 62/509,888, filed May 23, 2017, the entire contents of both of which are incorporated herein by reference.

### BACKGROUND OF THE DISCLOSURE

### 1. Field of Disclosure

Generally, exemplary embodiments of the present disclosure relate to the field of devices for ophthalmology, and in particular tonometry or tests to measure pressure inside an eye referred to as intraocular pressure (IOP). Exemplary implementations of certain embodiments of the present disclosure provide systems and methods for measuring eye pressure or tonometry and further provide a novel portable or hand held eye pressure sensor or tonometer.

### 2. Discussion of the Background of the Disclosure

Conventional methods for measuring IOP include Goldman tonometry, non-contact tonometry (NCT), electronic tonometry, and Schiotz tonometry, as generally described in “How Does Tonometry Eye Pressure Test Work?” by Troy Bedinghaus, OD (September 2016) attached hereto and made part of this disclosure as Appendix A (see also “Goldman Applanation Tonometry” by eyetec.net (opthalmictechnician.org 2015) attached hereto and made part of this disclosure as Appendix B).

All of these conventional tonometry techniques have various drawbacks.

For example, referring to FIGS. 8A-8D, a conventional Goldmann applanation tonometer 800 includes a housing 806 with a force adjustment knob 808, rod 804 protruding from housing 806, and head 805, which includes a split-image prism (or “biprism”) 802, mounted on rod 804. Tonometer 800 measures the force necessary to flatten an area of the cornea of 3.06 mm in diameter. At this diameter, the material resistance of the cornea of eye 810 to flattening is counterbalanced by the capillary attraction of the tear film meniscus to the tonometer head 805. Furthermore, the IOP (in mm Hg) equals the flattening force (in grams-force) multiplied by 10. FIG. 8A illustrates basic features of the tonometer 800, shown in contact with cornea of a patient's eye 810 and the direction of an observer's (for example a doctor or examiner) view 812.

Referring to FIG. 8B, the enlargement 814 shows the tear film meniscus 816 created by contact of the split-image prism 802 and cornea 811. A split-image prism 802 allows the examiner to determine the flattened area 816 with great accuracy. Referring to FIG. 8C, the view through the split-image prism 802 reveals circular 820 meniscus 818, which is converted into two semicircles 821 and 822 by the prisms 802. To outline the area of flattening 816, topical anesthetic and fluorescein dye are instilled in the tear film.

Referring to FIG. 8D, fluorescein semicircles, or mires, 821 and 822 visible through the split-image prism 802 move with the ocular pulse within an observer's view 812. The endpoint is reached when the inner edges of the semicircles 821 and 822 touch each other at the midpoint of their excursion as shown in the enlargement 814, which also depicts excursions 826 of the mires 821 and 822 caused by ocular pulsations. By properly aligning the mires 821 and 822, the examiner can ensure the appropriate area of corneal applanation and obtain a correct IOP reading. However, achieving such proper alignment, including proper width and position of the mires, is a very skilled task subject to human error.

On the other hand, NCT or “air puff” test can be inaccurate. Typically measurements from three “puffs” are averaged. However, the patient may feel discomfort and pull away from the machine during the air puffs, thus varying the distance from machine to eye surface which impacts the measurement accuracy. While Goldman tonometry is considered to be more accurate than NCT, it is much more invasive requiring anesthetic drops and fluorescein dye instilled into the eyes, and a probe that applies pressure on the cornea. Unlike Goldman tonometer which is not portable, electronic tonometry provides a handheld tonometer that looks like a pen, but like Goldman tonometer requires direct application to the cornea and is not considered as reliable as Goldman tonometry. Schiotz tonometry uses as indentation tonometer which determines pressure by measuring the depth of deformity caused by a small metal plunger applied directly to the cornea.

Presently, clinical methods that do not rely on instruments, for example when instruments are not available, allow patients to keep their eyes closed such that a skilled physician uses the thumb and index finger to ballot the eye and pick up a high pressure by touch.

A conventional tonometer that can measure IOP though the eyelid is described in “Transpalpebral Tonometer for Intraocular Pressure Measuring,” by A. P. Nesterov at www.diaton-tonometer.com/products/tonometer-diaton/articles (2017) attached hereto and made part of this disclosure as Appendix C. However, when using this tonometer the position with respect to the eye is critical, because it relies on “dynamic (ballistic) way of dosated mechanical influence on the eye for evaluating its elastic peculiarities” (see Id.), and any deviation from required position can cause erroneous results.

### SUMMARY OF THE DISCLOSURE

Exemplary embodiments of the present disclosure address at least such drawbacks by providing systems and methods including an implementation where a patient's eyelids are closed and a hand held instrument has at least two sensors in contact with the eye at the same time such that instantaneous or historic pressure topography or wave on the eye and the firmness of the eye can be measured and recorded.

An exemplary embodiment of the present disclosure provides a device for measuring IOP including a sensing section comprising at least first and second sensors, a microprocessor, a sensor support, and a handle. The first sensor comprises a contact-sensitive surface that makes contact with the eye during the measuring procedure to determine the area of the eye surface in contacted with the sensing section. The second sensor comprises a force detector to determine the force applied by the eye surface when contacted by the sensing section.

According to an exemplary implementation, a microprocessor, for example disposed in the handle of the device, can receive essentially simultaneous input from the first and second sensors. Alternatively, or in conjunction with, first and second sensors can output time-tagged data that can be correlated to determine measured contact surface area and applied force at any given time. In yet another implementation, data taken at various frequencies over a time period can be interpolated and/or extrapolated to facilitate correlation of measurements as needed.

According to another exemplary embodiment of the present disclosure, a device for measuring IOP can also include an internal memory for storing measured data obtained by the first and second sensors.

According to an exemplary implementation, a device for measuring IOP can include a wired or wireless transmitter for outputting data obtained by the first and second sensors essentially in real time, or on demand, for example in batches at certain pre-set intervals or on command.

According to yet another exemplary embodiment of the present disclosure, an IOP measuring system and method can include an IOP measuring device, data storage internal to the device, or external, for storing instantaneous and/or historic data obtained by the IOP measuring device, and internal or external display system for visual output, for example in a graphical format, of processed real time and/or historical data obtained by first and second sensors.

According to still further exemplary embodiment of the present disclosure, an IOP measuring device, system, or method provide a sensing section comprising a plurality of contact-sensitive sub-subsections and a plurality of force-sensing sub-section. A microprocessor (internal and/or external to the device, in direct, wired and/or wireless communication with the sensing section and/or with an internal and/or external memory storing data obtained by the sensing section) can be configured to process measured data and output, for example a 3D or color-coded graph to show IOP pressure over the eye surface in contact with the sensing section.

According to an exemplary implementation of the present disclosure, depending on the type and number of contact sensors and force sensors employed in the sensing section, a method for determining IOP can include any or all of: normalization of collected measured data to obtain a single value for the IOP measurement; generation of a two-dimensional graphical representation of IOP versus contact area; generation of a surface map or 3D graph of pressure across the eye surface in contact with the sensing section. As described with reference to other embodiments, a desired visual graphic or numeric output of raw or processed measures data obtained by sensing section can be performed in real-time and/or as post processing of historic data. In exemplary implementation, the output can be continuous so as to show in real-time, and/or historically, changes in the measurements as a function of time.

In yet further exemplary implementations of the embodiments of the present disclosure, evaluation of the results of IOP measurements can be performed with reference to a predetermined standard value, graph, or map of pressure value and/or values. Alternatively and/or in conjunction with comparison to a predetermined standard, patient's own historical data obtained by IOP measurements according to embodiments of the present disclosure can be used as a reference, or to create a patient's baseline, to evaluate the IOP measurements. In still further exemplary implementation, any such evaluation can be performed essentially in real time as IOP measurements are obtained and/or during post-processing of measured data.

Systems, methods and IOP measuring devices provided according to exemplary embodiments of the present disclosure can perform IOP measurement by direct contact of sensing section to eye cornea, or by contact of sensing section to the eyelid thereby avoiding discomfort of most conventional IOP measuring devices and techniques.

Furthermore, according to embodiments of the present disclosure, since the contact surface area is also considered and evaluated as part of the measuring process, position of IOP measuring device on the eye surface is taken into account.

Another exemplary embodiment of the present disclosure provides a device and methodology including one or more features a Goldman-type tonometer and further comprising a sensor, for example an annular sensor, for detecting corneal contacted area and outputting an indication thereof to an observer to facilitate determination of when the endpoint is reached, such that a certain selected or predetermined area of cornea has been flattened, to obtain correct IOP reading.

According to an exemplary implementation, a camera and/or an image sensor can be provided for real-time video streaming or still frame photo imaging of retina as the cornea flattens during measurement procedure.

In yet further exemplary implementation, real-time or post processing of image data can be performed, for example by an internal or an external microprocessor, to determine when the endpoint is reached to obtain correct IOP reading.

In still further exemplary implementation, an internal or external monitor can be provided for viewing video and/or image output of the camera and/or image sensor.

In yet another exemplary implementation, image and/or sensor data can be transmitted via wired and/or wireless communication to an external data storage and/or processing devices, including but not limited to cloud-based data storage and/or virtual computing media.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings, wherein

FIG. 1A is an illustrative conceptual diagram showing diagrammatic representation of IOP measuring device according to an exemplary implementation of exemplary embodiments of the present disclosure with respect to a human eye.

FIG. 1B is an illustrative conceptual diagram showing diagrammatic representation of IOP measuring device according to another exemplary implementation of exemplary embodiments of the present disclosure.

FIGS. 2A, 2B, 2C, 3A, 3B, 3C, 4A, 4B, and 4C provide a diagrammatic illustration of application of a sensing section according to exemplary implementations of exemplary embodiments of the present disclosure to a surface of an eye to perform an IOP measurement procedure, and an exemplary representation of output data or information from IOP measuring device according to exemplary implementations of exemplary embodiments of the present disclosure.

FIG. 5 is another illustrative example of information and/or data collected and/or computed and output and/or displayed according to exemplary implementations of exemplary embodiments of the present disclosure.

FIG. 6 is an illustrative block diagram illustrating showing a diagrammatic representation of a system according to an exemplary embodiment of the present disclosure including an IOP measuring device according to exemplary implementations of exemplary embodiments of the present disclosure.

FIGS. 7A and 7B are illustrative conceptual diagram showing diagrammatic representation of an IOP measuring device according to yet another exemplary implementation of exemplary embodiments of the present disclosure.

FIGS. 8A, 8B, 8C, and 8D conceptually illustrate an example of a conventional Goldman tonometer and operation thereof.

FIGS. 9A, 9B and 9C are illustrative conceptual diagrams showing features and operation of an IOP measuring device according to exemplary implementations of still another exemplary embodiment of the present disclosure.

### DETAILED DESCRIPTION OF EXEMPLARY EMBODIMENTS

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the described disclosure. Also, descriptions of well-known functions and constructions are omitted for clarity and conciseness.

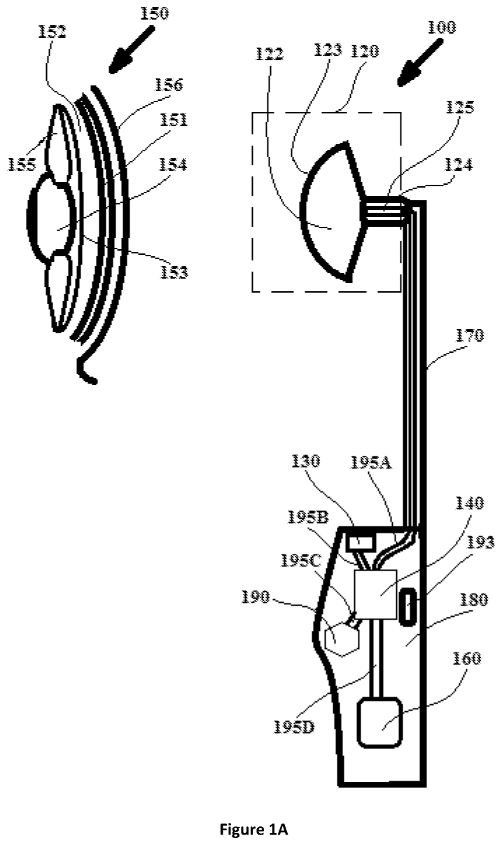

FIG. 1A (see also FIG. 1B) is a block diagram showing diagrammatic representation of IOP measuring device 100 according to exemplary embodiments of the present disclosure with respect to a human eye 150, whose well known anatomy includes cornea 151, anterior chamber 152, iris 153, pupil 154, and posterior chamber 155. Eye lid 156 is also shown since embodiments of the present disclosure provide for IOP measurement by direct contact with exterior surface of cornea 151 and/or by contact with exterior surface of eye lid 156.

Referring to an example of FIG. 1A, exemplary implementations of embodiments of the present disclosure provide an IOP measuring device 100 that includes a sensing section 120 with a first sensor 122 and a second sensor 124, a controller 130, a microprocessor 140, input/output (I/O) device(s) 160 such as wired and/or wireless transceiver and/or one or more communication port(s), a sensor support 170, and a handle 180. The first sensor 122 comprises a contact-sensitive surface 123 that makes contact with the eye 150 during the IOP measuring procedure, as explained in more detail below, to determine the area of the eye surface in contacted with the sensing section 120. The second sensor 124 comprises a force detector 125 to determine the force applied by the eye surface when contacting the eye by the sensing section 120. Similarly, FIG. 1B is a block diagram showing a diagrammatic representation of IOP measuring device 100A according to another exemplary embodiments of the present disclosure which includes section 120A in an essentially linear configuration with sensor support 170 and handle 180 (instead of an essentially 90-degree configuration of FIG. 1A). In the description that follows, any reference to a device having an essentially 90-degree configuration (as in FIG. 1A) is likewise applicable to a device having an essentially linear configuration (as in FIG. 1B).

IOP measuring device 100 can also include memory 190, which can be internal or external to microprocessor 140. Memory 190 can also comprise a portable removable memory such as USB or a flash drive. Any and/or all communication, such as 195A, 195B, 195C, and 195D, between any and/or all electronic components of IOP measuring device can be wired or wireless depending on the configuration of respective devices and other factors such as cost, portability, reliability, etc.

Power to various components, such as microprocessor 140 and/or I/O device(s) can be provide by an internal or external power source 193 which can include, for example, a battery (disposable or rechargeable).

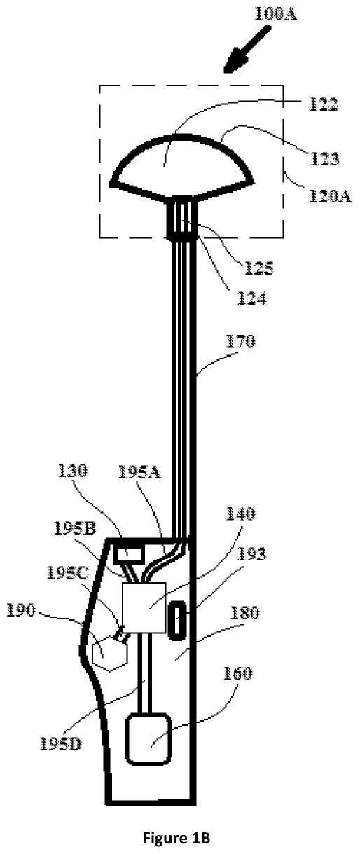

Methods of performing IOP measurements and operation of IOP measuring device and systems according to exemplary embodiments of the present disclosure are described with reference to FIGS. 2A-2C, 3A-3C and 4A-4C which provide a diagrammatic illustration of application of a sensing section 120 to a surface 250 of an eye 150 to perform an IOP measurement procedure, and an exemplary representation of output data or information from IOP measuring device 100. As note previously, according to embodiments of the present disclosure, surface 250 can be an exterior surface of cornea 151 or eye lid 156. While contact area of surface 250 is illustrated as being essentially circular, any shape of the contact area is within the scope of the present disclosure.

Referring to FIGS. 2A-2C, 3A-3C and 4A-4C, data output 200 of sensing section 120 according to an exemplary implementation can be represented in two-dimensional, X-Y axis, plot 206 of contact area (Y-axis) 202 for example in units of square millimeter (mm²) versus pressure (X-axis) 204 for example in units of millimeter mercury (mmHg). FIGS. 2A, 3A, and 4A provide a diagrammatical illustration of a side view of sensing section 120 of IOP measuring device 100 with respect to eye surface 250 of eye 150. FIGS. 2B, 3B, and 4B provide a diagrammatical illustration of contact-sensitive surface 123 of first sensor 122 from the perspective of the eye 150. FIG. 2C, 3C and 4C show an exemplary output 200 of IOP measuring device 100 before or during the IOP measuring process according to exemplary embodiments of the present disclosure.

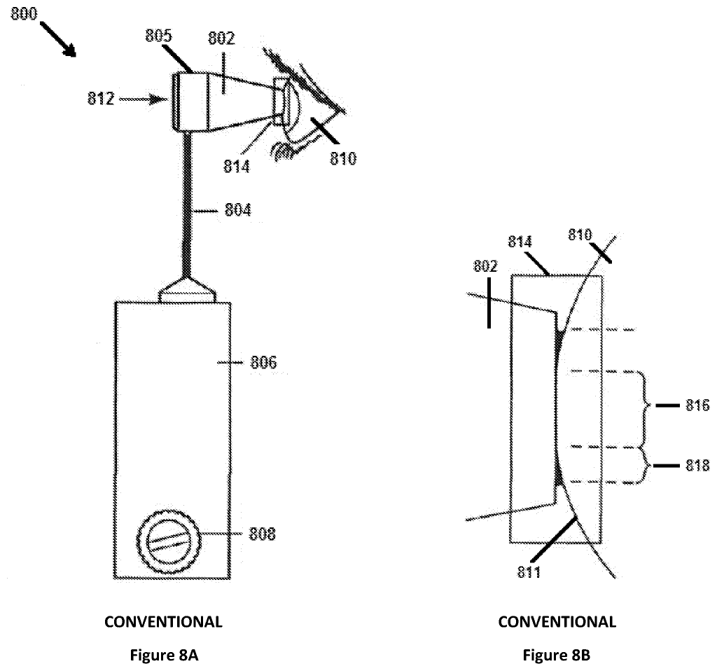

In an exemplary implementation, a contact area 260,262, or 255,256, can be calculate based on interaction with eye surface 250 sensed by a contact-sensitive surface 123 of first sensor 122 at a time t, and pressure can be calculated based on force 258, 259 applied by eye surface 250 sensed by force detector 125 of second sensor 124 at the same time t. In an exemplary implementation, these calculations can be performed by microprocessor 140 and stored in memory 190 for real time output during the IOP measuring procedure, or historic download during or after IOP measuring procedure, via I/O device 160. In yet further exemplary implementation, output, activation of components, and other functions such as ON/OFF, can be controlled by a controller 130 which can include an interactive interface (such as simple switches and/or complicated touch screen displays) for receiving input from the user of IOP measuring device 100 and providing visual, audible, and or tactile output to the user. In still further exemplary implementation, controller 130 can receive and process external commands for example via wired or wireless communication with a user station (such a desktop, laptop, or personal display device (PDA)) 610, as illustrated in FIG. 6.

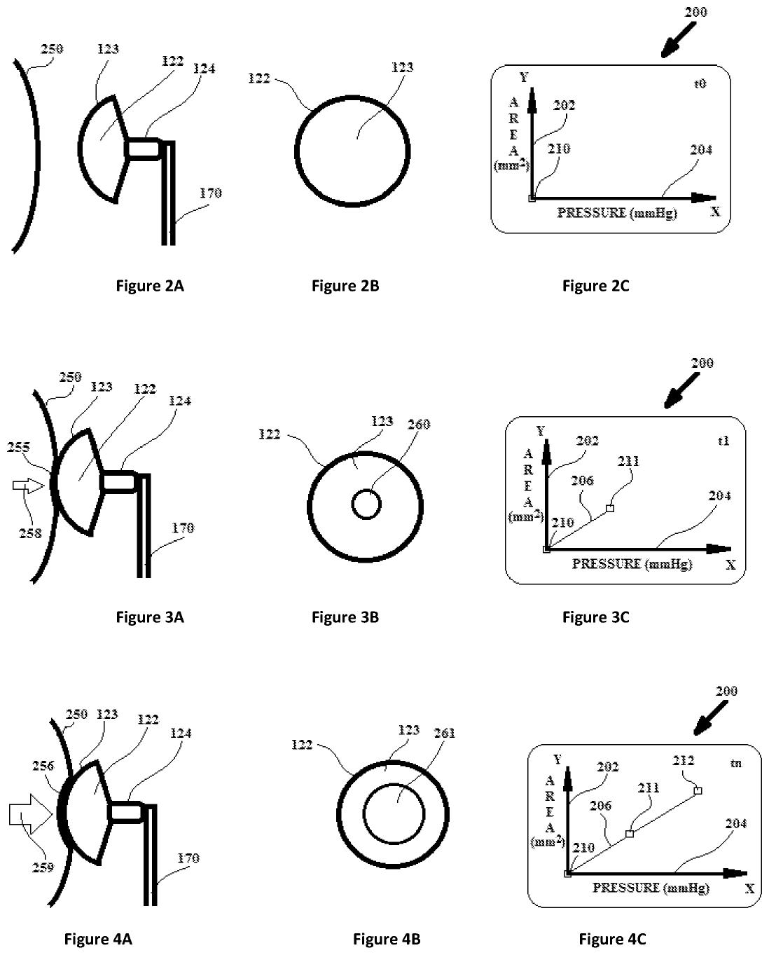

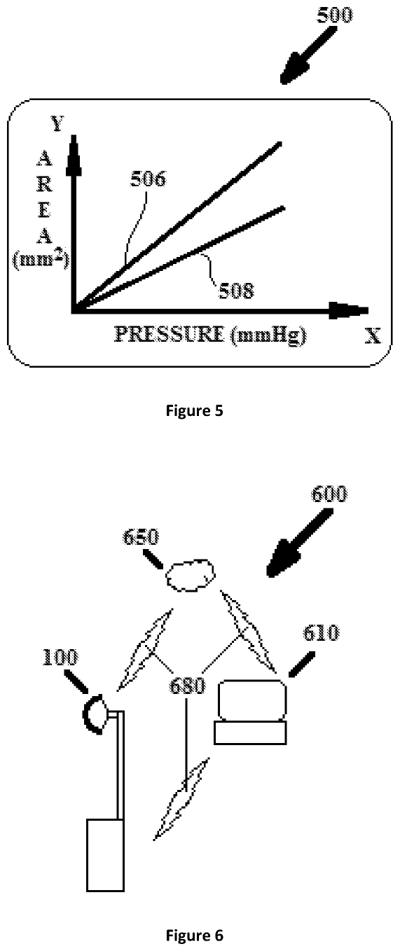

Referring to FIGS. 2A, 2B and 2C, prior to application of sensing section 120 to eye 150 (for example at time t0) data output 200 is illustrative 210 of no contact between sensing section 120, in particular contact surface 123 of first sensor 122, and the eye surface 250. As the IOP measuring device 100 indents the eye 150 at time t1 during the IOP measuring procedure, as shown in the example of FIGS. 3A, 3B and 3C, data output 200 is illustrative 211 of (1) Y-Axis contact area value—based on contact occurring over a portion 260 of contact-sensitive surface 123 at time t1 between sensing section 120 and portion 255 of eye surface 250, and (2) X-Axis pressure value—based on force 258 applied over portion 255 of eye surface 250 corresponding to portion 260 of contact-sensitive surface 123. As the IOP measuring device 100 further indents the eye 150 at time tn during the IOP measuring procedure, as shown in the example of FIGS. 4A, 4B and 4C, data output 200 is illustrative 212 of (1) Y-Axis contact area value increasing—based on increased indentation of eye surface 250 resulting in increased contact occurring over a portion 261 of contact-sensitive surface 123 at time tn between sensing section 120 and portion 256 of eye surface 250, and (2) X-Axis pressure value increasing—based on increased force 259 applied over portion 256 of eye surface 250 corresponding to portion 261 of contact-sensitive surface 123. The pressure that is exerted by application of IOP measuring device 100 to indent a given area of the cornea correlates with the IOP pressure.

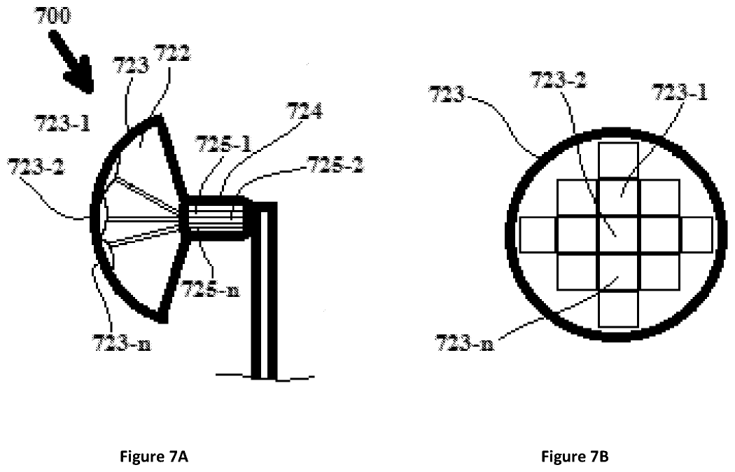

In an exemplary implementation of the present disclosure, IOP measurements taken during a procedure would produce a unique graph or data for the eye undergoing the IOP measuring procedure. Referring to FIG. 5, such measurement could be compared and evaluated with respect to other measurements, or a baseline, as illustrated by measurements taken during two IOP measuring procedures 506 and 508 (as noted, in an exemplary implementation graph 508 could be a baseline graph) where graph 506 may be illustrative of an eye with diagnosed IOP pressure of 20 mmHg, while graph 508 may be illustrative of an eye with diagnosed IOP pressure of 25 mmHg. A softer eye would have a large area indent (or interacting with contact-sensitive surface 123) for a given pressure that a firm eye.

FIG. 6 is a block diagram illustrating an exemplary embodiment of the present disclosure providing a system 600 including IOP measuring device 100 in wired and/or wireless (e.g., intra- or internet based) communication 680 with: external work station 610, which can provide and receive control information to/from device 100, perform data processing and/or display and/or storage; and/or external data storage 650, which could be cloud-based, shared and/or secure. Likewise, work station 610 can be in wired and/or wireless (e.g., intra- or internet based) communication 680 with data storage 650.

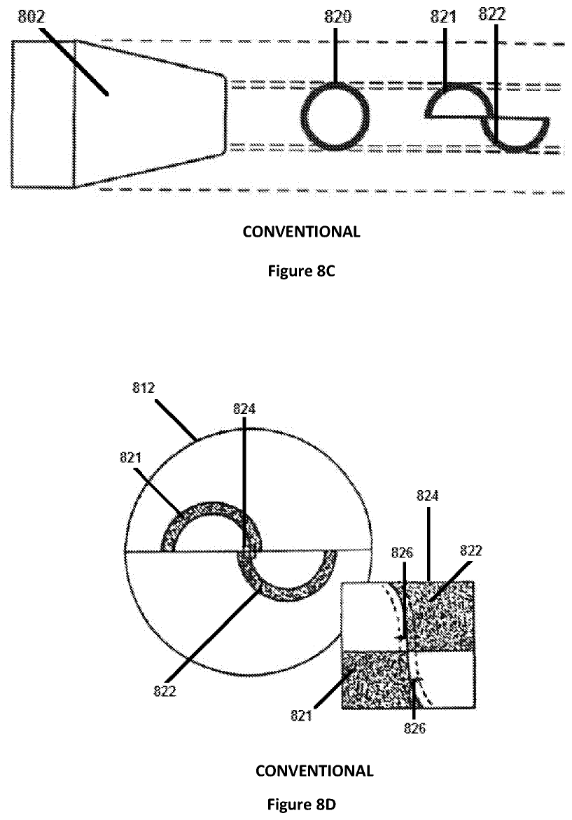

In yet another exemplary embodiment of the present disclosure illustrated in FIGS. 7A and 7B, IOP measuring device 700 can include a first sensor 722 comprising a contact-sensitive surface 723 with multiple contact-sensitive sub-areas 723-1, 723-2, . . . 723-n configured to sense corresponding contact pressure in conjunction with a second sensor 724 comprising a corresponding plurality of force detectors 725-1, 725-2, . . . 725-n detecting the force applied to the eye surface at each of the corresponding contact-sensitive sub-areas 723-1, 723-2, . . . 723-n. These measurements can be processed by an internal microprocessor of IOP measuring device 700, or by an external desk top such that of system 600 to compute a single, (for example normalized based on measurements from all sub-areas) value of pressure at time t of the IOP measurement, or produce a topological graph based on pressure values sensed in all sub-areas over contact sensitive surface. In an exemplary implementation, such graph could also be a 3D graph of pressure (Z-axis) with respect to a given contact-sensitive sub-area location (X-Y axis). The resolution of such a graphical representation would be directly related to the number of contact-sensitive sub-areas provided on contact-sensitive surface 723.

In yet another exemplary implementation, all or any portion of the measured data can be interpolated or extrapolated to produce a smoother graphical representation.

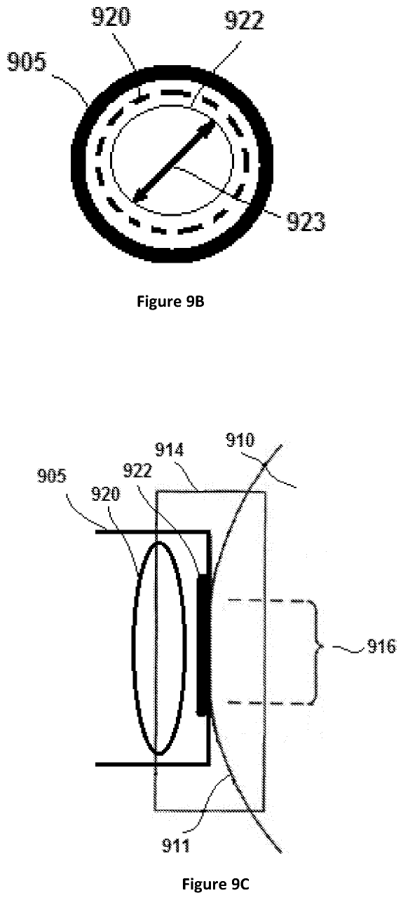

Referring to an example of FIGS. 9A-9C, another exemplary embodiment of the present disclosure provides a tonometer 900, which can be used in applanation tonometry where the cornea 911 is flattened and IOP is determined by measuring the applanation force (F) and the area flattener (A) according the formula IOP=F/A. According to an exemplary implementation, tonometer 900 includes a housing 906 with a controller 908 configured to receive user input and adjust the force accordingly, such as a force adjustment knob, or a slide, or touch sensitive pad, or voice a controller, a rod 904 protruding from housing 906, and head 905 mounted on rod 905. Head 905 includes a sensor 922, for example an annular circular sensor have a preset sensing diameter 923 (such as 3.06 mm) receptive to contact with a surface of cornea 911 and outputting an indication when the corneal contact area, or flattened area, 916 is essentially equal to the preset sensing diameter 923. For example, sensing tonometer 900 can measure the force necessary to flatten an area of the cornea of 3.06 mm in diameter by setting sensing diameter 923 to 3.06 mm and outputting an indicating that the measurement endpoint has been reach when preset diameter 923 has been sensed and/or by automatically detecting the amount of force applied and outputting computer IOP accordingly. FIG. 9A diagrammatically illustrates tonometer 900, shown in contact with cornea of a patient's eye 910.

Referring further to FIG. 9A, according to an exemplary implementation, an image processing device, such as a camera, 924 and/or an image sensor or a lens 920 can be provided for real-time video streaming or still frame photo imaging of retina as the cornea flattens during measurement procedure.

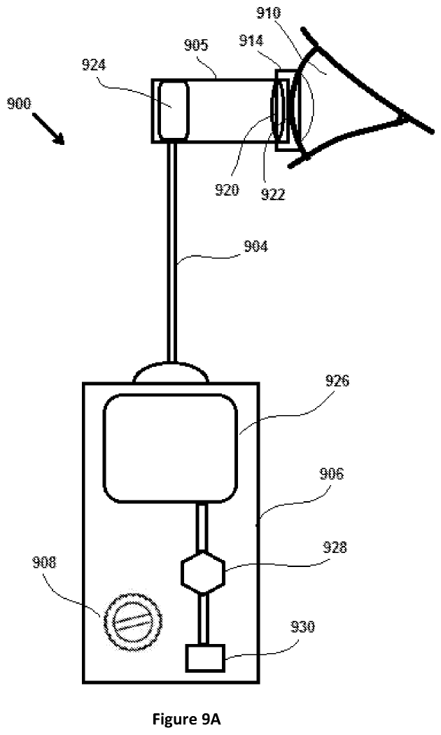

In another exemplary implementation, real-time or post processing of image data obtained by camera 924 and/or image sensor 920 can be performed, for example by an internal microprocessor 928 or an external microprocessor (not shown) with respect to a selectively preset or input reference frame, to determine when the endpoint is reached to obtain correct IOP reading. For example, diameter of tear film meniscus, such as 820 illustrated in FIG. 8C, created by contact of the image sensor or lens 920 and cornea 911 can be determined by processing the image as the cornea flattens and compared to a desired preset value, such as 3.04 mm. Such implementation can avoid the use of a sensor 922.

In yet another exemplary implementation, image sensor or lens 920 can be replaced by a split-image prism, such as biprism 802, and real-time or post processing of image data obtained by camera 924 can be performed, for example by an internal microprocessor 928 or an external microprocessor (not shown), to determine when the endpoint is reached to obtain correct IOP reading. For example, an image from the split-image prism (of a circular meniscus created by contact of the split image prism and cornea 911 converted into two semicircles, such as 821 and 822 of FIG. 8C, as the cornea flattens) can be processed to determined relative position of inner edges of said two semicircles with respect to each other and, when the inner edges of the two semicircles touch each other at the midpoint of their excursion, compute IOP of said eye as a function of said force applied, as illustrated in FIG. 8D. Such implementation can also avoid the use of a sensor 922.

In still further exemplary implementation, an internal monitor 926 or an external monitor (not shown) can be provided for viewing video and/or image output of the camera 924 and/or image sensor, or lens, or split-image prism 920.

In yet further exemplary implementation, image processing device, such as a camera, 924 can be configure for wired and/or wireless communication with any of: an internal monitor 926 or an external monitor (not shown), an internal microprocessor 928 or an external microprocessor (not shown), an external data storage and/or processing devices, including but not limited to cloud-based data storage and/or virtual computing media.

In yet another exemplary implementation, image and/or sensor data can be transmitted, via a transceiver 930 configured for wired and/or wireless communication, to an external data storage and/or processing devices, including but not limited to cloud-based data storage and/or virtual computing media.

While the present disclosure has been shown and described with reference to certain exemplary embodiments thereof, it will be understood by those skilled in the art that various changes in form and details may be made therein without departing from the spirit and scope of the present disclosure.

Other objects, advantages and salient features of the disclosure will become apparent to those skilled in the art from the details provided, which, taken in conjunction with the annexed drawing figures, disclose exemplary embodiments of the disclosure.

Those of skill in the art further appreciate that the various illustrative logical blocks, modules, circuits, and algorithm steps described in connection with the embodiments disclosed herein may be implemented as electronic hardware, computer software, or combinations of both. Skilled artisans may implement the described functionality in varying ways for each particular application, but such implementation decisions should not be interpreted as causing a departure from the scope of the present disclosure. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

The above-presented description and figures are intended by way of example only and are not intended to limit the illustrative embodiments in any way except as set forth in the appended claims. It is particularly noted that various technical aspects of the various elements of the various exemplary embodiments that have been described above can be combined in numerous other ways, all of which are considered to be within the scope of the disclosure.

Accordingly, although exemplary embodiments have been disclosed for illustrative purposes, those skilled in the art will appreciate that various modifications, additions, and substitutions are possible. Therefore, the disclosure is not limited to the above-described embodiments, but may be modified within the scope of appended claims, along with their full scope of equivalents.

I claim:

1. A device for measuring IOP pressure comprising:
a head section comprising a contact surface configured to apply a force to an essentially circular portion of an eye surface;
a contact sensors configured at said contact surface of said head section; and
a microprocessor in communication with said contact sensor,
wherein
said contact sensor makes contact with said essentially circular portion of said eye surface when said head section applies said force to determine when a diameter of said essentially circular portion of said eye surface corresponds to a predetermined value, and
said microprocessor computes IOP of said eye as a function of said force applied when said essentially circular portion of said eye surface corresponds to said predetermined value, and outputs said computed IOP.

2. The device of claim 1, wherein said contact sensor comprises an annular circular sensor.

3. The device of claim 1, further comprising:
a housing; and
a rod protruding from said housing and having said head section dispose on a portion of said rod protruding from said housing.

4. The device of claim 1, wherein said microprocessor is configured in said housing.

5. The device of claim 1, further comprising an image sensor disposed in said head section and configured to receive images at least of said essentially circular portion of said eye surface.

6. The device of claim 5, further comprising an image processor in communication with said image sensor and outputting at least one of a video stream and a still photo of said images received by said image sensor.

7. The device of claim 6, wherein said image sensor is in communication with said microprocessor.

8. The device of claim 5, further comprising a monitor in communication with said image sensor to display said images received by said image sensor.

9. The device of claim 6, further comprising a monitor in communication with at least one of said image sensor and said image processor to display at least one of said video stream and said still photo of said images received by said image sensor.

10. The device of claim 1, further comprising a controller configure to receive user input to adjust said force applied to said eye surface.

11. A device for measuring IOP pressure comprising:
a head section comprising a contact surface configured to apply a force to an essentially circular portion of an eye surface;
an image sensors configured in said head section to receive an image at least of said essentially circular portion of said eye surface and to output data representative of said image; and
a microprocessor in communication with said image sensor,
wherein said microprocessor
receives said data representative of said image when said head section applies said force,
processes said data with respect to a selectively preset or input reference frame to determine when a diameter of said essentially circular portion of said eye surface corresponds to a predetermined value,
computes IOP of said eye as a function of said force applied when said essentially circular portion of said eye surface corresponds to said predetermined value, and
outputs said computed IOP.

12. The device of claim 11, further comprising an image processor in communication with said image sensor and outputting at least one of a video stream and a still photo of said images received by said image sensor.

13. The device of claim 11, further comprising a monitor in communication with said image sensor to display said images received by said image sensor.

14. The device of claim 12, further comprising a monitor in communication with at least one of said image sensor and said image processor to display at least one of said video stream and said still photo of said images received by said image sensor.

15. The device of claim 11, further comprising a controller configure to receive user input to adjust said force applied to said eye surface.

16. A device for measuring IOP pressure comprising:
a head section comprising a contact surface configured to apply a force to an essentially circular portion of an eye surface;
a split-image prism configured in said head section with respect to said contact surface to receive an image at least of said essentially circular portion of said eye surface and to convert said received image of said essentially circular portion into a split image comprising two semicircles of said received image;
an image processor configured in said head section to receive said split image and to output data representative of said split image; and
a microprocessor in communication with said image sensor,
wherein said microprocessor
receives said data representative of said split image when said head section applies said force,
processes said data to determined relative position of inner edges of said two semicircles with respect to each other,
computes IOP of said eye as a function of said force applied when inner edges of said two semicircles touch each other at the midpoint of excursion of said two semicircles, and
outputs said computed IOP.

17. The device of claim 16, further comprising a monitor in communication with at least one of said image sensor and said image processor to display at least one of said video stream and said still photo of said images received by said image sensor.

18. The device of claim 16, further comprising a controller configure to receive user input to adjust said force applied to said eye surface.
